(19) United States
(12) Patent Application Publication
(10) Pub. No.: US 2016/0362706 A1
(43) Pub. Date: Dec. 15, 2016

(54) METHODS AND SYSTEMS FOR PRODUCING PRODUCTS USING ENGINEERED IRON OXIDIZING BACTERIA AND COPPER METAL

(71) Applicant: THE TRUSTEES OF COLUMBIA UNIVERSITY IN THE CITY OF NEW YORK, New York, NY (US)

(72) Inventors: Alan West, Tenafly, NJ (US); Scott Banta, Fairfield, CT (US)

(73) Assignee: THE TRUSTEES OF COLUMBIA UNIVERSITY IN THE CITY OF NEW YORK, New York, NY (US)

(21) Appl. No.: 15/248,426

(22) Filed: Aug. 26, 2016

Related U.S. Application Data
(63) Continuation-in-part of application No. PCT/US2015/037125, filed on Jun. 23, 2015.
Application No. PCT/US2015/037125 is a continuation-in-part of application No. 14/444,527, filed on Jul. 28, 2014.
(60) Provisional application No. 62/015,890, filed on Jun. 23, 2014.
(60) Provisional application No. 62/210,036, filed on Aug. 26, 2015.

(51) Int. Cl. C12P 1/04; C12N 15/74; C12N 9/04; C10L 1/00; C12N 9/88

(52) U.S. Cl. CPC C12P 1/04; C10L 1/00; C12N 9/88; C12N 9/0006; C12Y 401/01072; C12Y 101/01001; C12N 15/74; C10L 2290/26; C10L 2200/0469; C10L 2290/42

(57) ABSTRACT

Methods and systems for producing a particular biofuel or a particular chemical using genetically modified iron-oxidizing bacteria (IOB) and copper as a redox mediator are disclosed. In some embodiments, the methods include the following: providing an IOB that have been genetically modified to enable them to generate the particular biofuel or the particular chemical; feeding a first source of ferrous iron to the IOB; feeding water, carbon dioxide, and oxygen to the IOB; producing one of the particular biofuel and the particular chemical, ferric iron, and an IOB biomass; providing a first source of copper metal; solubilizing the first source of copper metal to produce cupric ions and reduce the ferric iron to ferrous iron; electrochemically reducing the cupric ions to produce a second source of copper metal; feeding ferrous iron reduced from the ferric iron to the IOB; and feeding the second source of copper metal to the IOB.

### CROSS REFERENCE TO RELATED APPLICATION(S)

This application is a continuation-in-part of International Application No. PCT/US2015/037125, filed Jun. 23, 2015, which claims the benefit of U.S. Provisional Application No. 62/015,890, filed Jun. 23, 2014, and is a continuation-in-part of U.S. application Ser. No. 14/444,527, filed Jul. 28, 2014, which was a U.S. National Stage application of International Application No. PCT/US2012/026697, filed Feb. 27, 2012, and this application also claims the benefit of U.S. Provisional Application No. 62/210,036, filed Aug. 26, 2015, each of which is incorporated by reference as if disclosed herein in its entirety.

### STATEMENT REGARDING FEDERALLY SPONSORED RESEARCH OR DEVELOPMENT

This invention was made with government support under contract numbers 0206-1565, DE-AR0000060, and DE-AR0000553 awarded by the Department of Energy. The government has certain rights in the invention.

### BACKGROUND

There has been interest in the development of liquid biofuels as these processes have the potential to directly fix carbon dioxide into transportation fuels, which is potentially carbon neutral and politically attractive. Cellulose based biofuels including bioethanol, algae-derived lipids, cyanobacteria, and algae derived hydrogen (H₂) are among the most studied biofuels. Despite the promise of these technologies and processes, there are specific limitations that preclude their wide-spread application. For example, post-processing of algal cells and derived lipids imposes higher production costs on algal biodiesel. The production rates of H₂ from cyanobacteria still remains low and productivity needs to be improved. Genetically engineered photosynthetic organisms have also been explored for bioethanol production. However separation of ethanol from the aqueous phase remains a challenge.

Microbial fuel cells have been under investigation and development for more than a century, as the use of cells to harvest electrical energy from waste streams is attractive for many reasons. In a bio fuel cell, biological catalysts are used on an anode to oxidize biofuels, and a cathode is created that can use the generated electrons to reduce oxygen to water. These systems can either be microbial with living cells on the electrodes, or they can be enzymatic systems, with purified enzymes on the electrodes. In both designs, power can be generated from the oxidation of bio fuels, and there are many advantages to these systems over conventional fuel cells and other power generation schemes. However, much research still needs to be done with microbial fuel cells to make them practical and cost-efficient. A significant limitation for both enzymatic and microbial fuel cells is the need for mediators to enable electrical contact between the biological components and inorganic electrode. In some microbial systems, these mediators are made by the organisms themselves, and in other technologies, synthetic mediators are added to the system. In some systems, cells must make physical contact with the electrodes for electron transfer. This can be a significant limitation as it reduces the cellular mass that can be used for biochemical conversion.

There is an intense global interest in the development of next-generation bio fuels. Currently, bio fuels are produced using the metabolic activities of heterotrophic organisms that transform organic materials, such as sugar, into fuels such as ethanol. Autotrophic bacteria have recently attracted attention as potential biosynthetic platforms for chemical production since autotrophs do not require organic compounds as substrates and thus do not involve agriculture. More recently, there has been interest in producing fuels and chemicals using electricity (termed electrofuels) and autotrophic bacteria such as Acidithiobacillus ferrooxidans (“A. ferrooxidans”), Nitrosomona europaea, Sporomusa ovata, and Ralstonia eutropha. A. ferrooxidans cells are an attractive candidate for this approach as they grow planktonically and the inorganic Fe³⁺ produced by the cells are readily reduced electrochemically. Thus, soluble iron is recycled between an electrochemical reactor and a bioreactor containing genetically engineered A. ferrooxidans cells. The cells grow continuously using electrochemically reduced iron while fixing gaseous CO₂. In these types of processes, it is desired that the oxidized iron is kept soluble at high concentrations.

As a means of making an electrofuels process economical, it is vital to control the capital expenditures of both the electrochemical and the bioreactor. Considerable efforts are devoted to developing engineering media that allow high efficiency and high production rates in both processes. This presents many challenges because of the limited solubility of ferric iron at pH values greater than two.

Biofuels are generated from bacteria that produce ethanol, diesel, and other petrochemicals from carbon sources. However, most biofuels are produced from plant biomass, which is inherently inefficient, requires large amounts of land, and competes with human food consumption. Alternatively, biofuels generated from iron-oxidizing bacteria (IOB) only require carbon dioxide and an electricity source. IOB oxidize ferrous iron to ferric iron while fixing carbon dioxide, and can be genetically modified to produce petrochemical fuels. By electrochemically reducing ferric iron, IOB are resupplied with ferrous iron to continue the iron oxidation and fuel production process. Unfortunately, the poor solubility of ferric iron ultimately limits the efficiency of this method for fuel generation.

### SUMMARY

Living cells have the ability to reproduce and maintain their catalytic machinery, and their metabolic pathways can be rationally altered to meet desired process objectives. But efficient electron transfer from the electrode to the organism can limit metabolic production, and the use of mediating species can result in a process that is not economically viable. One way to address these limitations is to explore alternative organisms that naturally utilize mediators that are more attractive. The disclosed subject matter includes the metabolic engineering of chemolithoautotrophic IOB, such as A. ferrooxidans, to develop a process that can overcome these limitations. IOB have the natural ability to fix carbon dioxide while oxidizing ferrous iron (Fe²⁺) to ferric iron (Fe³⁺).

Referring to FIG. 1, aspects of the disclosed subject matter include methods and systems for producing chemicals and fuels from dilute CO₂ enabled by copper as a redox mediator. This technology improves IOB biofuel production using copper as a reduction and oxidation mediator. Specifically, the IOB solubilize copper sulfide ores through ferrous iron oxidation to ferric iron, which oxidizes solid copper to cupric ions while also reducing ferric iron to ferrous iron. The regeneration of ferrous iron during copper solubilization allows IOB to continuously oxidize iron, fix carbon dioxide, and produce petrochemicals. Solid copper is provided either as feedstock from copper ores, or regenerated by electrochemical reduction of cupric ions to solid copper.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of methods and systems according to some embodiments of the disclosed subject matter;

FIG. 2 is a diagram of methods and systems according to some embodiments of the disclosed subject matter;

FIG. 3 is a diagram of methods and systems according to some embodiments of the disclosed subject matter;

FIG. 4 is a diagram of methods and systems according to some embodiments of the disclosed subject matter; and

FIG. 5 is a chart of a method according to some embodiments of the disclosed subject matter.

### DETAILED DESCRIPTION

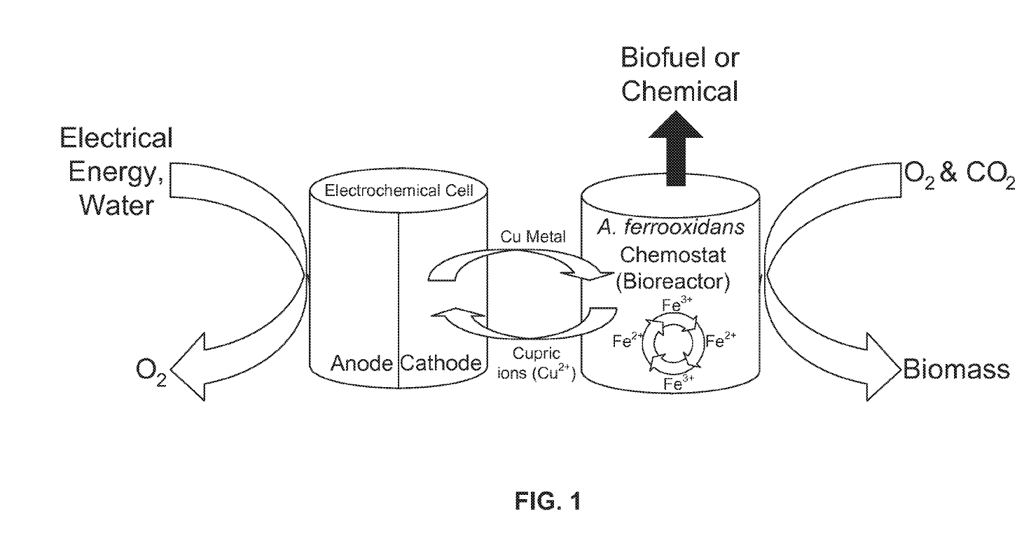

Referring again to FIG. 1, aspects of the disclosed subject matter include methods and systems that include the application of chemolithoautotrophic IOB for concomitant carbon dioxide fixation, conversion of the carbon dioxide to a product such as a biofuel or a chemical, and oxidation of ferrous iron to ferric iron. The ferric iron produced upon ferrous iron oxidation is reacted with copper metal to continually reduce it back to ferrous iron, which allows continuous operation of the process and production of the products. Additional ferrous iron can be added from other ferrous iron-rich sources, as necessary. Metabolic engineering is used to introduce a new pathway into the bacteria that starts with the precursors for amino acid synthesis to create particular biofuels or chemicals.

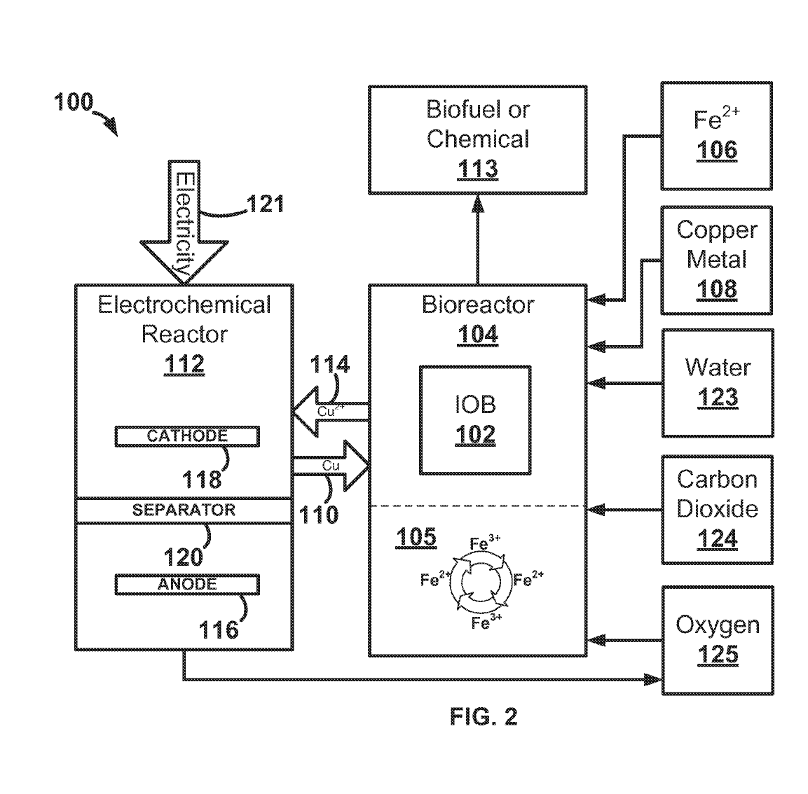

Referring now to FIGS. 2-4, some embodiments include systems and methods for producing products such as biofuels and chemicals using genetically modified iron-oxidizing bacteria (IOB) and copper as a redox mediator for reducing ferric iron to ferrous iron. As shown in FIG. 2, some embodiments include a system 100 for producing particular biofuels and chemicals using genetically modified IOB 102 and copper metal as a redox mediator. IOB 102 are grown in a bioreactor 104 and fed ferrous iron and carbon dioxide. As IOB 102 oxidizes the ferrous iron to ferric iron, they produce a bio fuel or a chemical.

Copper metal is introduced to bioreactor 104 to provide electrons to IOB 102 and to react with the ferric iron, which reduces the ferric iron to ferrous iron and generates cupric ions (Cu²⁺). The ferrous iron is recycled within bioreactor 104 and oxidized by IOB 102. In some embodiments, system 100 includes a copper reaction chamber 105, which is positioned within bioreactor 104, where the copper metal is reacted with ferric iron to generate ferrous iron and cupric ions. In some embodiments, copper reaction chamber 105 is positioned in a region of bioreactor 104 that is substantially depleted of dissolved oxygen so as to inhibit the auto-oxidation of the copper metal via reaction with acid. As discussed below, in some embodiments, copper reaction chamber 105 is positioned outside of bioreactor 104. Carbon dioxide is used as a base material to be fixed into a biofuel or chemical. Initially, ferrous iron is typically provided from a first source 106 that is external to system 100, e.g., a ferrous iron-rich stream in fluid communication with bioreactor 104. In some embodiments, the ferrous iron used in system 100 is substantially provided by a second source (not shown) that is generated by an electrochemical reactor (also not shown). The ferrous iron is oxidized to ferric iron by IOB 102 and reduced back to ferrous iron by the copper metal in a continuous process. Additional ferrous iron, after the initial amount fed to bioreactor, is introduced to the bioreactor as necessary. The copper metal is typically initially provided by a first source 108 that is external to system 100, e.g., purchased or otherwise provided bulk copper metal. When copper metal reacts with the ferric iron, the ferric iron is reduced to ferrous iron and cupric ions (Cu²⁺) are formed. In some embodiments, copper metal used in system 100 is substantially provided by a second source 110 that is generated in an electrochemical reactor 112 where the cupric ions formed while reducing the ferric iron to ferrous iron are electrochemically reduced to form copper metal. The copper metal serves as a mediator for transferring electrons to IOB 102. As discussed later, in some embodiments, substantially all of the copper metal used by bioreactor 104 is provided by a source external to system 100.

Bioreactor 104 includes IOB 102 that have been genetically modified to include a particular metabolic pathway to enable them to generate a particular product 113, i.e., a biofuel or chemical. The operating parameters of bioreactor 104 are typically optimized to maximize the consumption of ferrous iron, the production of cupric ions 114, and the production of product 113. In some embodiments, product 113 is a leachate for solubilizing a target substance, such as a source of metal, as will be discussed in greater detail below. Methods and systems according to the disclosed subject matter have operating conditions that are optimized for optimal yield, conversion, etc. Bioreactors included in methods and systems according to the disclosed subject matter are typically operated in a continuous flow mode to maximize the conversion of the substrates to the products.

Cupric ions 114, which are generated in bioreactor 104, are introduced to electrochemical reactor 112, which is in fluid communication with the bioreactor. Electrochemical reactor 112 includes electrodes, i.e., an anode 116 and a cathode 118, a separator 120, and source of electrical energy 121. In some embodiments, cathode 118 is formed substantially from nickel or glassy carbon and anode 116 is formed from materials known in the art. In some embodiments, flow through or flow by porous electrodes are used. In some embodiments, separator 120 is a cation selective membrane, to allow for proton transfer across the membrane to achieve acid balance.

Electrochemical reactor 112 is typically configured to electrochemically reduce cupric ions 114 to second source 110 of copper metal using source of electrical energy 121. In system 100, cupric ions 114 will be continually regenerated back to copper metal, i.e., second source 110, ferric iron will be continually reduced back to ferrous iron and oxidized by IOB 102, and the recycle loop can be theoretically closed without the need for additional copper metal input from first source 108 or ferrous iron input from first source 106 beyond startup.

System 100 includes a source of water 123, a source 124 of carbon dioxide, and a source of oxygen 125, all of which are in fluid communication with bioreactor 104. In some embodiments, oxygen produced in electrochemical reactor 112 is recycled for use in bioreactor 104. In some embodiments, source 124 is carbon dioxide removed from air or energy plant emissions. In some embodiments, either in place of or in addition to carbon dioxide, carbonate, e.g., from mineral sources, is fed to bioreactor 104.

Although not included in FIG. 2, in some embodiments, system 100 includes pumps for pumping the various constituents, into, thru, and out of the system. In addition, the pumps are typically programmable to allow electrochemical reactor 112 to be turned off when the price of electricity is high and turned on when the price is low. Also, the pumps typically include a separator unit to separate one or more particular constituents that are to be pumped to other components of system 100 from the other constituents.

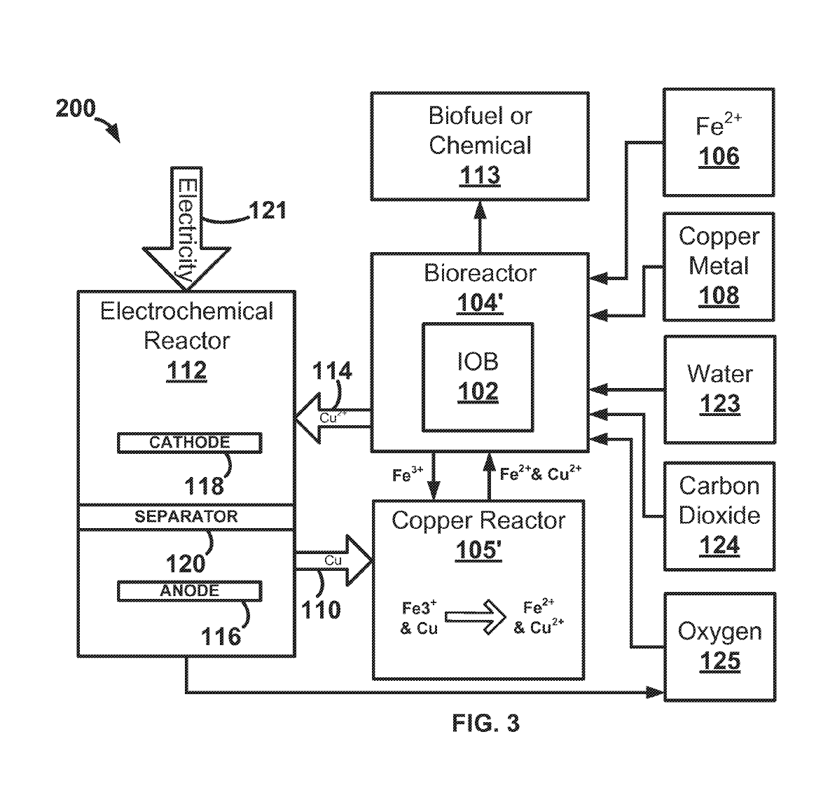

Referring now to FIG. 3, as mentioned above with respect to system 100, some embodiments include a system 200 where a copper reaction chamber, i.e., copper reactor 105′, is positioned outside of, but in fluid connection with, bioreactor 104′. Because bioreactor 104′ typically is an oxygen-rich, acidic environment that promotes the auto-oxidation of copper metal and limits the desirable reaction of copper metal with ferric iron to reduce the ferric iron to ferrous iron, system 200 separates copper reaction chamber 105′ from bioreactor 104′ and optimizes both independently. System 200 ensures that the copper metal will react with and reduce the ferric iron to ferrous iron, which can be recycled back to bioreactor 104′. The cupric ions generated can then be electrochemically reduced to copper metal and recycled back to bioreactor 104′. In some embodiments, copper reaction chamber 105′ includes copper foil, copper sheets, copper particles, or a combination thereof.

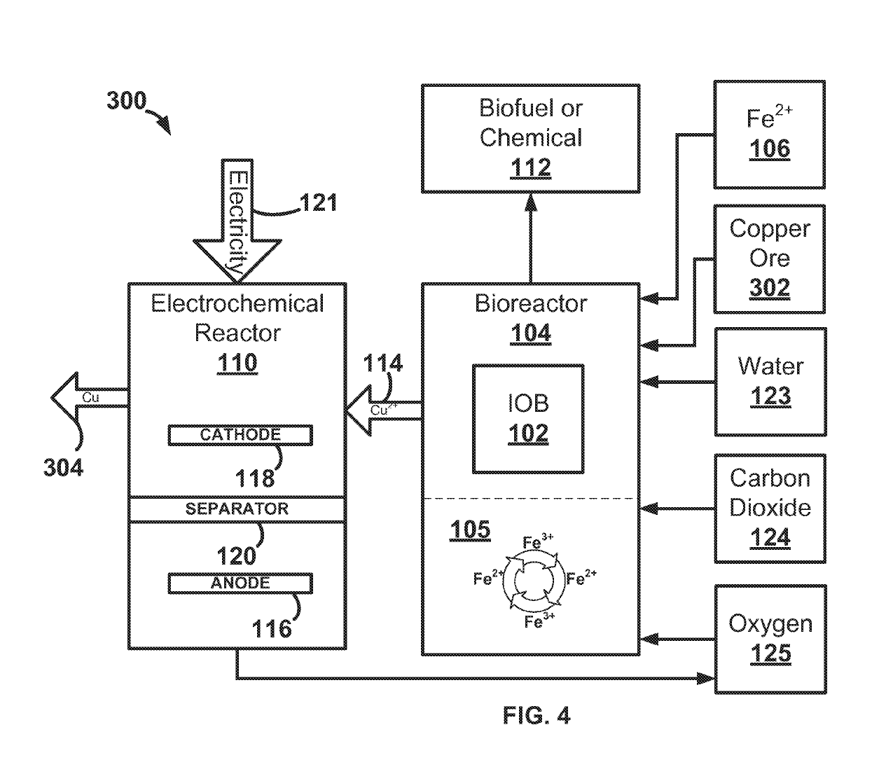

As shown in FIG. 4 and also referred to above, some embodiments include a system 300 where copper metal is introduced in the form of copper-containing ore 302. As IOB 102 oxidizes ferrous iron to ferric iron, copper metal in the copper ore is solubilized to cupric ions. In some embodiments, product 113 is a leachate which contributes to this solubilization. In some embodiments, solubilization utilizing the leachate comprising bringing the leachate into contact with a target substance, such as with copper ore. In some embodiments, the IOB are in spatial proximity to the target substance to help facilitate contact between the target substance and the leachate. Similar to systems 100 and 200, the cupric ions are electrochemically reduced to produce copper metal 304. Unlike systems 100 and 200, in system 300, copper metal 304 produced is typically sold as a commodity rather than recycled to bioreactor 104. Instead, additional copper-containing ore 302 is added to bioreactor 104. Although system 300 is shown to include copper reaction chamber 105 positioned inside bioreactor 104, similar to system 100, in some embodiments, system 300 may include a copper reaction chamber that is positioned outside of the bioreactor, similar to system 200 (not shown).

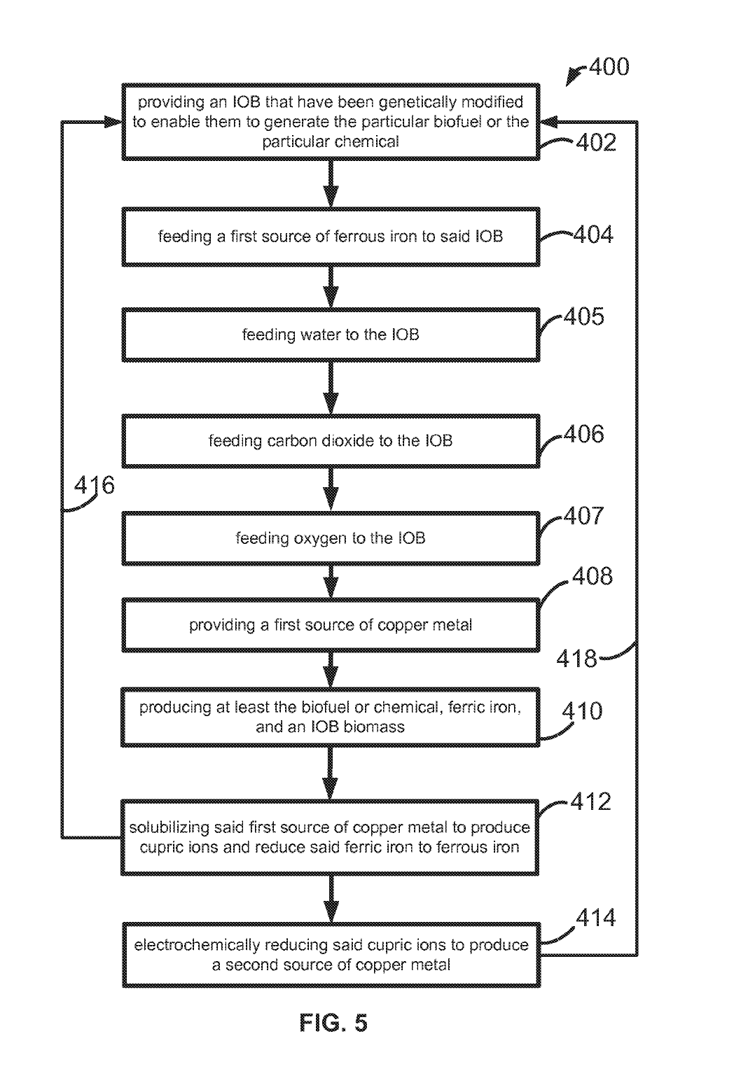

Referring now to FIG. 5, some embodiments include a method 400 for producing a biofuel or chemical using genetically modified IOB and copper metal as a redox mediator. As shown in FIG. 5, at 402, IOB that have been genetically modified to include a particular metabolic pathway to enable them to generate a particular biofuel or chemical are provided in a bioreactor.

In some embodiments, the IOB is substantially A. ferrooxidans, e.g., wild type A. ferrooxidans 23270 strain or similar, and the IOB are genetically modified by including at least one of a 2-keto-acid decarboxylase gene (outlined by box) and an alcohol dehydrogenase gene or similar. The production of isobutanol in prokaryotic hosts begins with the amino acid biosynthesis pathways. These pathways produce 2-keto acids, and these are converted to aldehydes using a 2-keto-acid decarboxylase. Alcohol dehydrogenase is then used to convert the aldehydes to alcohols. In the case of isobutanol, the valine biosynthesis pathway is used, and the starting precursor is 2-keto-isovalerate. In some embodiments, the IOB are genetically modified to include at least one of an acyl-ACP reductase and an aldehyde-deformylating oxygenase.

Still referring to FIG. 5, at 404, a first source of ferrous iron is fed to the IOB. At 405, water is fed to the IOB. At 406, carbon dioxide is fed to the IOB. At 407, oxygen is fed to the IOB. At 408, a first source of copper metal, e.g., bulk copper metal or copper ore, is introduced to the bioreactor. At 410, depending on the IOB used, a particular biofuel or chemical, ferric iron, and an IOB biomass are produced as the IOB oxidized the ferrous iron while fixing carbon dioxide. At 412, the first source of copper metal is solubilized as it reacts with the ferric iron to produce cupric ions and reduce the ferric iron to ferrous iron. At 414, the cupric ions are electrochemically reduced to produce a second source of copper metal. At 416, the ferrous iron produced at 412 is fed to the IOB. At 418, the second source of copper metal is fed to the IOB or otherwise provided to the bioreactor. After step 418, method 400 operates in a substantially continuous cycle where steps 404 and 408, i.e., the addition of ferrous iron and copper metal, are only repeated as necessary. In some embodiments, the biofuel is one of isobutanol, a long chain alcohol, or an alkane. In some embodiments, the particular chemical is one of a commodity chemical, a specialty chemical, feedstock such as an acid, an amino acid, an organic acid, a carbohydrate, or a combination thereof.

Some embodiments of the disclosed subject matter include methods and systems that do not include the electrochemical regeneration of copper metal. For example, where a feed rich in copper metal exists, e.g., locally available copper-containing ore, the conversion of ferrous iron and CO₂ to a valuable product (biofuel or other chemical) can be achieved without electrochemical regeneration of copper metal. Instead, the cupric ions generated may be electrochemically reduced to form copper metal that can later be sold.

The high tolerance of IOB for copper, along with copper's higher solubility compared to iron, improves biofuel production efficiency. In addition, this technology can be used with existing biomining equipment, reducing capital and development costs. By combining genetically modified IOB with copper biomining, this technology produces biofuels from carbon dioxide and electricity using a copper source.

Methods and systems according to the disclosed subject matter have, but are not limited to, the following benefits over known technologies:

1. Increased production rates in bioreactor;

2. Exploitation of the organism in their “natural chemical environment,” for which they have evolved tolerance to otherwise toxic substances;

3. The ability to use an existing, commercial, large scale electrochemical process with minimal development costs;

4. Reductions in bioreactor cost; and

5. The ability to piggyback off existing industrial infrastructure.

Methods and systems according to the disclosed subject matter may be used in integrated copper extraction from copper ores with electrofuel generation to offset carbon dioxide emissions and fuel consumption in mining operations. Methods and systems according to the disclosed subject matter can be used in copper extraction from metal alloys and scrap metals for recycling. In methods and systems according to the disclosed subject matter, bacteria can be optimized for a carbon dioxide capture and sequestration system instead of fuel production. In methods and systems according to the disclosed subject matter, petrochemical fuel or other small molecule production from carbon dioxide can be generated.

In some embodiments, the present disclosure is directed to the use of genetically altered biomining organisms that secrete a leachate, thus enhancing material dissolution. In some embodiments, the leachate is an organic acid or other compound. In some embodiments, the organic acid is isobutyric acid. In some embodiments, the compound is heptadecane. In some embodiments, two heterologous biosynthetic pathways are expressed in the biomining organisms. In some embodiments, species other than A. ferrooxidans are modified to produce the leachate. In some embodiments, wild-type cells are genetically modified to produce the modified organisms (cells) of the present invention. In some embodiments, previously-modified cell lines are further modified to produce the modified cells of the present invention.

A 7-fold improvement in productivity of isobutyric acid was obtained through improved media formulations in batch cultures. Steady-state efficiencies were lower in continuous cultures, likely due to ferric inhibition. If coupled to solar panels, the photon-to-fuel efficiency of this proof-of-principle process approaches estimates for agriculture-derived biofuels. These efforts lay the foundation for the utilization of this organism in the exploitation of electrical energy for biochemical synthesis.

Methods and systems according to the disclosed subject matter may be used for production of fuels, commodity chemicals, or specialty chemicals. It may be attractive in locations where a source of agricultural feedstock is not readily available. In some embodiments, the present disclosure is directed to a method of extracting a target substance from a material. In some embodiments, the target substance is a metal. In some embodiments, the target metal is copper. In some embodiments, the target metal is a metal other than copper, such as a precious metal, a rare earth metal, and the like. In some embodiments, the material is naturally occurring. In some embodiments, the naturally occurring material is chalcopyrite. In some embodiments, the material is man-made. In some embodiments, the man-made material is electronic waste.

The production of bio fuels and commodity chemicals from agricultural feedstock is inherently inefficient, relies on extensive land use, and competes with food. Furthermore, methods and systems according to the disclosed subject matter may allow for integration of liquid fuel and electricity generation markets, solving a major problem with integration of large-scale renewable generation with the grid. Finally, this technology allows for the utilization of captured CO₂ as a carbon source. It thus may compete with carbon storage technologies.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method for producing a particular bio fuel or a particular chemical using genetically modified iron-oxidizing bacteria (IOB) and copper as a redox mediator, said method comprising:
providing an IOB that have been genetically modified to enable them to generate the particular biofuel or the particular chemical;
feeding a first source of ferrous iron to said IOB;
feeding water, carbon dioxide, and oxygen to said IOB;
providing a first source of copper metal;
producing at least one of said particular biofuel and said particular chemical, ferric iron, and an IOB biomass;
solubilizing said first source of copper metal to produce cupric ions and reduce said ferric iron to ferrous iron; and
electrochemically reducing said cupric ions to produce a second source of copper metal.

2. The method according to claim 1, further comprising:
feeding ferrous iron reduced from said ferric iron to said IOB; and feeding at least a portion of said second source of copper metal to said IOB.

3. The method according to claim 1, wherein said biofuel is one of isobutanol, a long chain alcohol, or an alkane.

4. The method according to claim 1, wherein said IOB is genetically modified to include a 2-keto-acid decarboxylase gene and an alcohol dehydrogenase gene.

5. The method according to claim 1, wherein said IOB is substantially A. ferrooxidans.

6. The method according to claim 1, wherein said particular chemical is one of a commodity chemical, a specialty chemical, a feedstock such as an acid, an amino acid, a carbohydrate, an organic acid, and a combination thereof.

7. The method according to claim 1, wherein after feeding a first source of ferrous iron, a first source of copper metal, water, carbon dioxide, and oxygen to said IOB, said method includes a substantially continuous cycle including the following steps:
producing at least one of said particular biofuel and said particular chemical, ferric iron, and an IOB biomass;
solubilizing said first source of copper metal to produce cupric ions and reduce said ferric iron to ferrous iron;
electrochemically reducing said cupric ions to produce a second source of copper metal;
feeding ferrous iron reduced from said ferric iron to said IOB;
feeding said second source of copper metal to said IOB; and feeding additional ferrous iron, a first source of copper metal, water, carbon dioxide, and oxygen to said IOB as necessary.

8. The method according to claim 1, wherein said first source of copper metal is copper-containing ore.

9. The method according to claim 1, wherein solubilizing said first source of copper metal to produce cupric ions and reduce said ferric iron to ferrous iron comprises contacting said particular chemical with said first source of copper metal.

10. The method according to claim 9, wherein the particular chemical is isobutyric acid.

11. A system for producing a particular bio fuel or a particular chemical using genetically modified iron-oxidizing bacteria (IOB) and copper as a redox mediator, said system comprising:
a bioreactor including IOB that have been genetically modified to include a particular metabolic pathway to enable them to generate at least one of the particular biofuel or the particular chemical;
a first source of ferrous iron in fluid communication with said bioreactor;
a source of water in fluid communication with said bioreactor;
a source of oxygen in fluid communication with said bioreactor;
a source of carbon dioxide in fluid communication with said bioreactor;
a first source of copper metal in fluid communication with said bioreactor;
a copper reaction chamber for reacting said copper metal with ferric iron to generate ferrous iron and cupric ions;
an electrochemical reactor in fluid communication with said bioreactor, said electrochemical reactor configured to electrochemically reduce said cupric ions to a second source of copper metal.

12. The system according to claim 11, wherein said copper reaction chamber is positioned within said bioreactor.

13. The system according to claim 12, wherein said copper reaction chamber is positioned in a region of said bioreactor that is substantially depleted of dissolved oxygen.

14. The system according to claim 11, wherein said copper reaction chamber is positioned outside of said bioreactor and is in fluid communication with said bioreactor and said electrochemical reactor.

15. The system according to claim 14, wherein said copper reaction chamber includes copper foil, copper sheets, copper particles, copper-containing ore, or a combination thereof.

16. The system according to claim 11, wherein said biofuel is one of isobutanol, a long chain alcohol, or an alkane.

17. The system according to claim 11, wherein said IOB is genetically modified to include a 2-keto-acid decarboxylase gene and an alcohol dehydrogenase gene.

18. The system according to claim 11, wherein said IOB is substantially A. ferrooxidans.

19. The system according to claim 11, wherein said particular chemical is one of a commodity chemical, a specialty chemical, a feedstock such as an acid, an amino acid, a carbohydrate, an organic acid, and a combination thereof.

20. The method according to claim 11, wherein the particular chemical is isobutyric acid.
